(19) United States
(12) Patent Application Publication
(10) Pub. No.: US 2006/0235434 A1
(43) Pub. Date: Oct. 19, 2006

(54) Disposable skin marker method and apparatus

(76) Inventors: Sheila Pavich, Naperville, IL (US); Erika L. Moore, Delavan, WI (US)

Correspondence Address: BARNES & THORNBURG, LLP, P.O. BOX 2786, CHICAGO, IL, 60690-2786, US

(21) Appl. No.: 11/106,094

(22) Filed: Apr. 14, 2005

(51) Int. Cl. A61B 17/00

(52) U.S. Cl. 606/116

(57) ABSTRACT

A one-time skin marking system and method. The system includes a marking implement with a hollow body portion and a marking tip extending therefrom. When liquid contained in the hollow body portion is introduced to the tip, the marking implement is used to mark a surgical site and then discarded. The marking liquid is indelible and has a volume not greater than an amount substantially equal to the liquid carrying capacity of the marking tip.

### BACKGROUND OF THE INVENTION

This invention relates to marking of surgical sites, and in particular to a one-time skin marking system and method of marking a surgical site.

For obvious reasons, it is important that surgical procedures are performed in the proper location on the patient undergoing surgery. There have been instances of surgeries being performed in the wrong location, which, not only being dangerous, also leads to substantial potential liability of the surgical team guilty of the error. Thus, precision in both location and procedure to be performed is mandatory.

Skin marking implements have been developed over the years, and the Food and Drug Administration actually maintains a registry of skin markers that can be used for marking surgical sites. While such markers, can, indeed, be used for marking the site, usually indelibly, the problem is that although it is intended that the markers be used once, because of the nature of the markers and the ease of their use, they can be used multiple times on multiple patients. That, however, can lead to infection control issues since the fibrous tips of markers not only transport ink to the skin, but also can transfer bacteria from the skin back to the marker, and if the marker is used again on another patient, that bacteria can then be transferred to the second patient. Thus, single use is a very desirable end result.

Hospitals and other health care providers have developed surgical site protocols. They have developed strict policies on surgical site protection and marking, and single use of marking implements. The problem remains, however, that although all good intentions are that the marking implements be used only once, their really is nothing in all modern marking implements that prevents their being used on two or more patients in succession, thus leading to continued infection control issues.

### SUMMARY OF THE INVENTION

The invention is directed to a one-time skin marking system, comprising a marking implement having a hollow body portion and a marking tip extending from the marking implement, with the marking tip being in fluid communication with the hollow body portion and the marking tip have a predetermined liquid carrying capacity. A marking liquid is contained in the hollow body portion, with the marking liquid having a volume not greater than an amount substantially equal to the predetermined liquid carrying capacity. A pressure-sensitive expedient is provided for delivering the marking liquid to the marking tip when the marking implement is to be used.

In accordance with the preferred form of the invention, the pressure-sensitive expedient comprises a frangible container in the hollow body portion. Preferably, that frangible container comprises a glass ampule. In another form of the invention, a frangible, fluid-impervious barrier is provided between the hollow body portion and the marking tip.

The marking liquid is an ink, and preferably is indelible so that once the skin is marked, the ink cannot be removed and will naturally disappear only as skin exfoliation occurs.

It is preferred that a package be provided for containing the marking implement. The package includes at least a flexible portion, which permits pressure to be applied through the package to the hollow body portion.

The purpose of the invention is to provide single use for the marking implement. To that end, the volume of marking liquid provided is necessarily small, and it is preferred that the liquid volume be up to ⅔ milliliters, which permits skin marking once the marking tip is activated, but which substantially prevents further use because the implement, once used, simply does not have remaining capacity to be used again.

The method of marking a surgical site according to the invention comprises the steps of selecting the surgical site, then selecting a one-time skin marking implement having a normally dry marking tip. The tip is then activated to moisten the marking tip with a marking liquid, and the surgical site is then marked with the now-moistened marking tip. Finally, the skin marking implement is disposed.

### BRIEF DESCRIPTION OF THE DRAWING

The invention is described in greater detail in the following description of examples embodying the best mode of the invention, taken in conjunction with the drawing figures, in which:

FIG. 1 is an elevational view of one form of the skin marking system according to the invention,

FIG. 2 is an elevational view of a second form of the skin marking system according to the invention, and

FIG. 3 is an elevational view of a protective package for holding the marking implement of FIGS. 1 and 2.

### DESCRIPTION OF EXAMPLES EMBODYING THE BEST MODE OF THE INVENTION

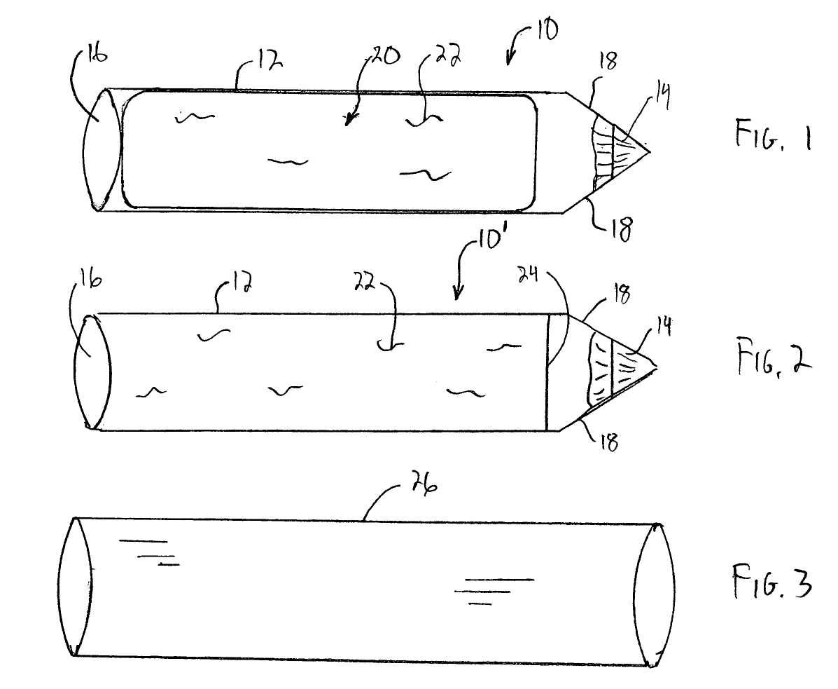

One form of a one-time skin marking system according to the invention is shown generally at 10 in FIG. 1. The skin marking system 10 is composed of two basis elements, a hollow body portion 12 and a marking tip 14 extending from one end of the hollow body portion 12.

The hollow body portion 12 is preferably formed of a flexible material, such as plastic, and includes a sealed end 16. The opposite end of the hollow body portion 12, from which the mark tip 14 extends, is tapered at 18, leading to an opening 20 through which the marking tip 14 extends.

The marking tip 14 is shown pointed and generally continuing the taper 18 of the hollow body portion 12, but the marking tip 14 can be formed in any desired shape for marking purposes. The marking tip preferably is made of a fibrous material which has a predetermined liquid carrying capacity and, once saturated with a marking liquid, can then release the marking liquid onto the patient being marked for surgical procedures.

In this form of the invention, a pressure-sensitive expedient in the form of a frangible container 20 is located within the hollow body portion 12. The frangible container 20 is preferably a glass ampule containing a liquid 22 therewithin. The liquid 22 is an ink, and preferably an indelible ink which, once applied to a patient, cannot then be removed. The ink is a medical grade ink used for skin marking purposes. The frangible container 20 is shown greatly exaggerated in relation to the size of the marking tip 14, simply for purposes of illustration. Actually the frangible container 29 is sized to contain a volume of marking liquid not greater than an amount substantially equal to the liquid carrying capacity of the marking tip 14. Thus, once the container 20 is fractured, all or substantially all of the liquid within the container 20 is absorbed in the tip 14 for marking purposes.

It has been found that an amount of the liquid 22 of approximately ⅔ milliliters is sufficient for marking purposes, and therefore the marking tip 14 needs to have approximately that liquid-carrying capacity. The tip 14 is therefore sized accordingly, depending on its liquid carrying capacity.

It is also important that not only must the marking tip 14 absorb the liquid 22 once released from the frangible container 20, but also the marking tip must be able to release enough of the liquid ink for marking of the patient when the skin marking system 10 is used. Therefore, not only must the material of the marking tip 14 have a sufficient liquid carrying capacity, but it also must have a sufficient liquid discharge capacity so that the surgical site being marked can be marked before the supply of liquid ink being released from the marking tip 14 is exhausted. However, the size of the marking tip 14 cannot be so great such that a great excess of liquid ink is retained after marking of a surgical site, so that the skin marking system could be for another patient. Thus, a balance must always be reached between the material of the marking tip 14 and the amount of liquid that can be released from the marking tip 14 for marking of a surgical site before the releasing capability is exhausted. Those skilled in the art will be able to determine the size of the marking tip 14 and the amount of liquid 22 to be absorbed by the marking tip depending on the ultimate writing capacity desired for one-time use.

A second form of a skin marking system according to the invention is shown at 10′ in FIG. 2. This form of the invention is identical to that of FIG. 1, except that the frangible container is omitted, and the liquid 22 is contained within the hollow body portion 12. A frangible, fluid-impervious barrier 24 is provided between the liquid 22 and the normally un-saturated marking tip 14. The barrier 24 can be any type of barrier which, once a certain pressure is exerted on the liquid 22 in the hollow body portion 12, fractures or separates in order to allow the liquid 22 to be introduced to the tip 14. Otherwise, the form of the skin marking system 10′ shown in FIG. 2 is identical to that of FIG. 1, and corresponding reference numerals have been used throughout.

For either form of the skin marking system 10 or 10′, preferably there also is provided a package 26 for storing the skin marking system 10 or 10′ before use. To allow activation, so that the liquid 22 is directed to the tip 14 when use is desired, the package 26 is preferably flexible, or includes a flexible portion so that pressure can be applied through the package 26 to the hollow body portion for activating the skin marking system 10 or 10′.

As explained above, preferably the volume of the liquid ink 22 is up to ⅔ milliliters, which provide a sufficient amount of liquid ink for marking of a surgical site, without providing too much so that an excess remains such that the marking system 10 or 10′ could be used again. Also, it is preferred, that the liquid ink 22 is such that once exposed to the atmosphere, the liquid portion of the ink readily evaporates, thus helping assure that if any excess remains in the marking tip 14 after use, the marking system 10 can be used only for one marking exercise.

In use, for marking a surgical site for location verification, first the site is selected. Then, the one-time skin marking implement 10 or 10′ according to the invention is selected, with the marking tip 14 initially dry. The skin marking implement 10 or 10′ is then activated to moisten the marking tip 14 with the marking liquid 22, and the medical practitioner then marks the surgical site with the now-moistened marking tip. Once the marking procedure has been completed, the medical personnel will then dispose of the skin marking implement 10 or 10′.

Due to the nature of the invention, and the small capacity of the marking tip 14 with the correspondingly-small amount of the liquid 22, the marking system according to the invention is truly single use. There is insufficient liquid remaining in the marking tip after use for one marking exercise to allow someone to reuse the skin marking system.

Various changes can be made to the invention without departing from the spirit thereof or scope of the following claims.

1. A one-time skin marking system, comprising
a. a marking implement having a hollow body portion,
b. a marking tip extending from said marking implement, said marking tip being in fluid communication with said hollow body portion and said marking tip having a predetermined liquid carrying capacity,
c. a marking liquid contained in said hollow body portion, said marking liquid having a volume not greater than an amount substantially equal to said predetermined liquid carrying capacity, and
d. a pressure-sensitive expedient for delivering said marking liquid to said marking tip.

2. The one-time skin marking system according to claim 1 in which said pressure-sensitive expedient comprises a frangible container in said hollow body portion.

3. The one-time skin marking system according to claim 2 in which said frangible container comprises a glass ampule.

4. The one-time skin marking system according to claim 1 in which said pressure-sensitive expedient comprises a frangible, fluid-impervious barrier between said hollow body portion and said marking tip.

5. The one-time skin marking system according to claim 1 in which said marking liquid is an ink.

6. The one-time skin marking system according to claim 5 in which said ink is indelible.

7. The one-time skin marking system according to claim 1 including a package for said marking implement.

8. The one-time skin marking system according to claim 7 in which said package includes at least a flexible portion permitting pressure to be applied through said package to said hollow body portion.

9. The one-time skin marking system according to claim 1 in which said volume is up to ⅔ milliliter.

10. A method of marking a surgical site for location verification, comprising the steps of
a. selecting the surgical site,
b. selecting a one-time skin marking implement having a normally dry marking tip,
c. activating the skin marking implement to moisten the marking tip with a marking liquid,
d. marking the surgical site with the now-moistened marking tip, and
e. disposing of the skin marking implement.

11. The method according to claim 10, in which step c includes applying pressure to a hollow body portion of the marking implement to force the marking liquid into contact with the marking tip.

12. The method according to claim 10, in which step c includes rupturing a frangible container in a hollow body position of the marking implement to introduce the marking liquid into contact with the marking tip.

13. The method according to claim 10, in which step b includes providing the skin marking implement with a volume of the marking liquid not greater than an amount substantially equal to liquid carrying capacity of the marking tip.

14. The method according to claim 10, in which step c includes the marking liquid being an indelible ink.
